(19) United States
(12) Patent Application Publication
(10) Pub. No.: US 2012/0296260 A1
(43) Pub. Date: Nov. 22, 2012

(54) DEVICE AND METHOD FOR PHOTODYNAMIC THERAPY

(76) Inventors: Freimut Vizethum, Rauenberg (DE); Reinhold Schuetze, Attnang-Puchheim (AT)

(21) Appl. No.: 13/377,250

(22) PCT Filed: Jun. 9, 2010
(86) PCT No.: PCT/EP2010/003457

§ 371 Date: Aug. 2, 2012

(30) Foreign Application Priority Data
Jun. 9, 2009 (DE) 10 2009 024 860.9

(51) Int. Cl. A61M 37/00

(52) U.S. Cl. 604/20

(57) ABSTRACT

The invention relates to an apparatus for photodynamic therapy and/or for destroying or reducing microorganisms, comprising an irradiation unit having at least one light source, by means of which a photosensitizer applied to a wound area to be treated is activated by way of irradiation, further comprising a camera for recording images of the wound, which is disposed in the irradiation unit, and a positioning unit, by means of which the irradiation unit can be oriented with respect to the wound area. The invention further relates to a method for operating such an apparatus. On a display for the images is a grid with visual indicia distinguishing fields to be irradiated by means of the light sources.

### BACKGROUND OF THE INVENTION

The invention relates to an apparatus for photodynamic therapy and/or for destroying or reducing microorganisms. The invention further relates to a method of using such an apparatus for photodynamic therapy and/or for destroying or reducing microorganisms.

WO 2005/035058 A1 discloses such an apparatus and a method, comprising an irradiation unit that can be moved by means of a positioning element. The irradiation unit contains several light sources by means of which a photosensitizer applied to the wound area to be treated is activated. The irradiation unit contains a camera by means of which images are taken of the wound area before carrying out, and while carrying out, the therapy. The light sources are designed in the form of a cluster lamp comprising several light-emitting diodes (LEDs) and are fixed in the irradiation unit by means of a cooler. Moreover, distance sensors are provided for monitoring the distance of the irradiation unit from the wound area.

Moreover, WO 2004/105 874 A2 discloses an apparatus which contains an irradiation unit having a light source that is notably designed as a laser. Using a light-activatable substance, in particular a dye, the microorganisms are sensitized and/or dyed and destroyed after being irradiated with light having a suitable wavelength and power density. The principle of action of photodynamic therapy (PDT) or antimicrobial photodynamic therapy (aPDT) is based on the physical action of energy transmission to the light-activatable substance, which is also referred to as a photosensitizer, following selective action and/or dyeing of the microorganisms, wherein the energy for the reactions is made available at the cell membrane. The energy generated by means of the light source of the irradiation device is focused on the microorganisms and the equilibrium positions of reactions that occur in the non-irradiated state in the “normal environment” are shifted and, as a consequence, the microorganisms are destroyed. The known apparatus contains an applicator, which can be coupled to the irradiation unit and comprises a light guide, wherein the free end of the applicator is guided as closely as possible to the area to be treated for the purpose of irradiating the same. This apparatus has been successfully applied in particular in dental medicine or in the mouth, jaw or facial areas. The known apparatus cannot be employed unconditionally for treating large-surface-area wounds, for example, or for the field of wound healing, by which is meant, merely by way of example, typical chronic wounds or skin ulcerations, such as wound ulcers which occur in patients that are no longer mobile in the tailbone region (decubitus), lower leg ulcerations caused by varicose veins or vascular obliterations (such as ulcus cruris), skin ulcers that can develop as a result of diabetes, such as diabetic foot syndrome (foot ulcer), or acutely infected wounds such as surgery wounds.

### SUMMARY OF THE INVENTION

Based on this, it is the object of the invention to provide an apparatus and a method for applying photodynamic therapy (PDT), or antimicrobial photodynamic therapy (aPDT), in wound healing, wherein in particular a reliable application and/or a proven microbe killing effect are achieved for a maximum possible wound area in a short time. An effective wound therapy that can be controlled in accordance with the respective circumstances is to be achieved with the lowest apparatus-related cost possible and/or with easy handling and/or with high functional reliability. The apparatus as well as the method should be easy to adapt to the medical and/or therapeutic requirements. The apparatus should be easy to adapt to the various positions on wounds, without major complexity, and it should be easy to adjust. Moreover, the weight of the irradiation unit should be specifiable at the lowest level possible, so that the positioning element can reliably maintain this unit in a position that is adjusted by the person performing the treatment. The mobility of the apparatus should be optimized, and notably a low weight and/or small dimensions should be achieved. Moreover, improved damping with respect to impacts or blows is to be achieved, so as to prevent damage to the light source and/or laser diodes.

The apparatus according to the invention and/or the method according to the present invention, or the use of the apparatus, allow a functionally reliable and practical application in the field of wound healing, while offering a simple design and/or easy handling. The therapy system according to the invention for applying PDT or aPDT in wound healing allows a reliable application and/or a proven optimal microbe killing effect in a short time, including, and particularly, for maximum possible wound areas. A dye, in particular a blue dye, and/or HELBO Blue Kutan contained in the photosensitizer is applied to the treatment area and/or the wound to be treated, or parts thereof. A defined time period is specified for the action of the photosensitizer, in particular at least 2 minutes, so that the dye molecules can bind to the microorganisms. Thereafter, excess dye is expediently rinsed off and/or dabbed off, wherein the following procedure is employed: aspirating the excess dye using a swab stick, passing over the therapy area with a swab saturated with physiological salt solution and NaCl, and finally aspirating the residual liquid using another swab stick. Thereupon, exposure to light having a suitable wavelength and energy is carried out. For this purpose, light having a wavelength of approximately 661 nm, a power density of at least 100 mW/cm² and an energy of at least approximately 3-5 J/cm² is preferably provided, and/or this is such that the photosensitizer is activated thus inducing the destruction of the microorganisms.

The at least one light source is movably disposed in the irradiation unit by means of a guide element, which is designed in particular as a linear guide, so as to be oriented to at least two different irradiation positions of the wound area. The wound area recorded by means of the camera is shown on a display, and the display further shows a grid, or the grid is superimposed on the camera image of the wound area that is displayed, wherein each field of the grid preferably corresponds to an irradiation field, notably in accordance with the respective positioning of the at least one light source that is disposed in the irradiation unit, by means of the guide element so as to be movable sequentially and/or consecutively from one irradiation field to the next irradiation field. A person performing the treatment can select and/or mark the fields to be irradiated by means of at least one light source. The display is advantageously part of an operating unit, which additionally contains buttons for scanning or generating the image of the wound area and for starting or stopping the irradiation, or other input keys. The operating unit moreover preferably contains display elements for the distance of the irradiation unit from the wound to be treated, or the body part to be treated, this distance being captured by means of distance sensors. The display, together with the buttons and the distance indicator, is preferably integrated in an operating unit, which expediently is disposed on the outside of the irradiation unit and/or designed as a separate unit that is easily accessible to the user.

The apparatus and/or the method and/or the therapy system according to the invention notably achieve the following advantages:
synergistically using presently available options with at least additively increased action,
reducing the overall costs of wound treatment because expensive dressings requiring frequent replacement can be dispensed with,
markedly increasing efficiency, in particular of the destruction of microbes,
sustainability, by way of saving expensive and limited resources, such as the silver in known dressings,
no development of resistance, as with classic systemic antibiotic therapies,
avoiding side effects of presently employed forms of therapy, such as the risk of triggering allergies or pain symptoms with silvers,
faster wound healing, whereby shorter hospital stays are achieved, especially in the case of chronic wound healing problems,
savings on drug costs and dressings,
overall economic advantages, in particular in the form of cost savings, for health care organizations and consequently for the entire health care system.
The apparatus according to the invention and/or the irradiation device substantially comprise the following components or modules, which will be described in more detail hereafter:
irradiation unit (irradiation head), including electronic or mechanical distance monitoring, wherein optionally, or preferably, an operating unit is contained,
power supply unit, which is optionally integrated in an equipment cart, including safety elements,
articulated arm and/or positioning arm for connecting the irradiation unit to an equipment cart, and
equipment cart, which carries and/or accommodates the entire unit.

The apparatus according to the invention and the method directed to the use thereof allow the PDT to be carried out to reduce pathogenic microbes (aPDT), while being easy to handle and offering high functional reliability, in particular for the treatment of the following skin wounds:
chronic wounds (ulcus decubitus)
acutely infected wounds (such as post-operative wounds)
wounds with local infections and
for inducing wound healing in stagnating wounds.

For this purpose, the following components or modules are employed:
light source/irradiation device
photosensitizer solutions, in particular HELBO Blue Kutan
aids such as applicators or swabs.

The elimination of—amongst others—the pathogenic microbes listed below is indicated and was verified by a clinical pilot study. A typical microbial spectrum in the wounds treated according to the invention comprises the following pathogens:

| | |
|---|---|
| Staphylococcus aureus | Bacteroides species |
| Escherichia coli | Proteus species |
| Enterococcus spp | group F Streptococcus |
| Streptococcus viridans | Staphylococcus schleiferi |
| Streptococcus agalactiae (B) | Enterobacter cloacae |
| Coagulase neg. Staph. | Streptococcus Pyogenes |

Pseudomonas aeruginosa
The therapy system according to the invention, which is also referred to as HELBO Wounds, essentially contains the following components:
dye (photosensitizer),
irradiation device and
optional aids, such as those taking the form of a sterile therapy set containing the dye as well as applicators, dabbing utensils, brushes and the like.

The photosensitizer employed is, in particular, the dye HELBO Blue Kutan, which has already received approval. This dye is a consumable and is packaged in individual doses, which are sufficient for treating wounds having the defined maximum wound area.

The name “Helbo” herein refers to the company for which the present invention was made, namely, Helbo Photodynamic Systems GmbH & Co., K.G.

The invention will be described in more detail hereafter based on the exemplary embodiments shown in the drawings, without thereby limiting the invention in this respect.

### BRIEF DESCRIPTION OF THE DRAWINGS

FIG. 1 shows the design of the overall system,

FIG. 2 shows a movably disposed light source,

FIG. 3 shows a configuration of the light source for spatial irradiation,

FIGS. 4-6 show views of the irradiation unit comprising the housing and articulated arm,

FIG. 7 shows an arrangement of three laser units of the irradiation unit or of the irradiation device,

FIG. 8 shows a view of the operating unit,

FIG. 9 shows an alternative configuration of the operating unit,

FIG. 10 shows the positioning of the irradiation unit over a wound on a lower leg,

FIG. 11 shows a flow chart for positioning the irradiation unit,

FIGS. 12-14 are illustrations of planar and spatial irradiation,

FIG. 15 shows a flow chart for positioning the pivotable light source or laser diode,

FIG. 16 is a camera image of a wound with an inserted grid in the display,

FIG. 17 shows a camera image according to FIG. 16 with marked, select irradiation fields,

FIG. 18 shows a flow chart for irradiation, including continuous distance control, and

FIGS. 19, 20 are camera images of the wound in the display with a grid and select fields in various irradiation positions.

### DETAILED DESCRIPTION OF THE INVENTION

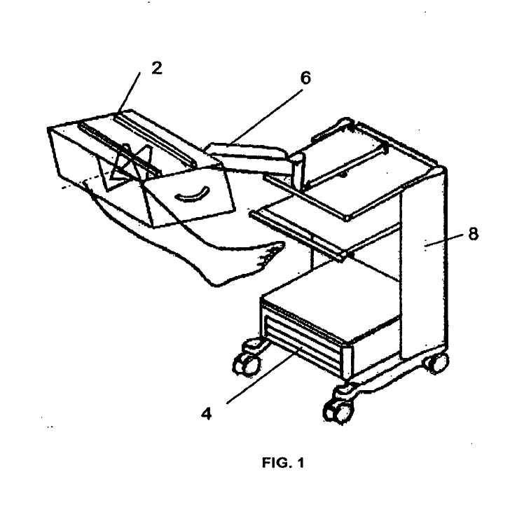

FIG. 1 shows the design of the overall system or therapy system, which is also referred to as HELBO Wounds and essentially includes the following components:
The light source and/or light sources employed, in particular for laser, light, can irradiate a predefined area with the required power density.
So as to be able to expose the total area of the treatment area to light within a predefined, preferably short, period of time, using a small number of light sources, and more particularly laser diodes, the light source and/or light sources according to the invention being moved in the irradiation unit from one irradiation position to the next by means of a guide element and a drive unit, with respective sub-areas being irradiated in each instance.
The irradiation device and/or the apparatus substantially consists of the following components, the detailed properties of which will be described hereafter:
irradiation unit 2 (irradiation head), including distance monitoring, which is done and/or designed electronically or mechanically, wherein additionally preferably an operating unit, which notably comprises a display, is integrated,
an operating unit,
a power supply unit 4, which preferably is integrated in an equipment cart and further preferably contains safety elements,
an articulated arm 6 and/or positioning arm and/or positioning element for connecting the irradiation unit 2 to the equipment cart 8, and
an equipment cart 8, which carries the entire unit.

The use of the HELBO Wounds system and/or execution of the method is provided for after wound cleaning and before applying a wound dressing. As an alternative, the use and/or execution of the method can be done depending on application studies and application observations.

A central part of the apparatus and the method is the configuration of the irradiation head and/or of the irradiation unit 2, notably using laser technology. The following is provided in order to achieve the required power data:
The light sources, and more particularly semiconductor laser diodes, are disposed in a linear or matrix shape, with the number of laser diodes being based on the output power of the types used. The irradiation is preferably carried out from three directions so as to spatially irradiate the therapy area to be treated and also to achieve the required high power density.

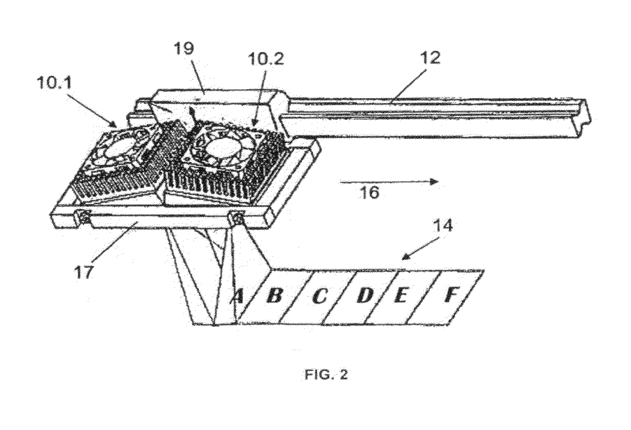

FIG. 2 shows a schematic design, wherein the light source and/or light sources 10.1, 10.2 and/or laser units are sequentially moved by means of a guide element and a drive unit along a guide rail 12, in particular in a linear manner, over irradiation positions 14 (A to F) in the direction of the arrow 16. No spatial irradiation is carried out with this variant. The two light sources 10.1 and 10.2 are disposed on a frame or carriage 17, which is part of the guide element and can be moved by means of a drive unit 19 along the guide rail 12 from one irradiation position to the next.

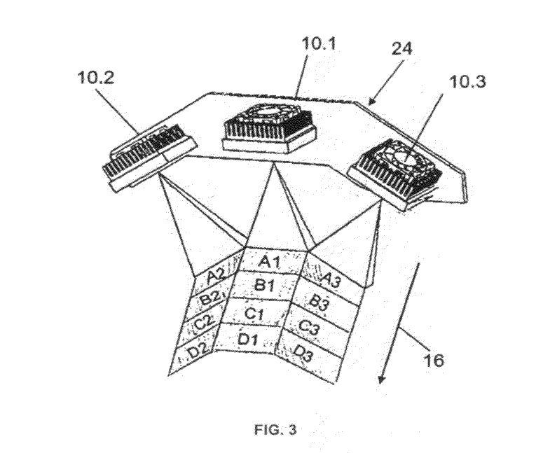

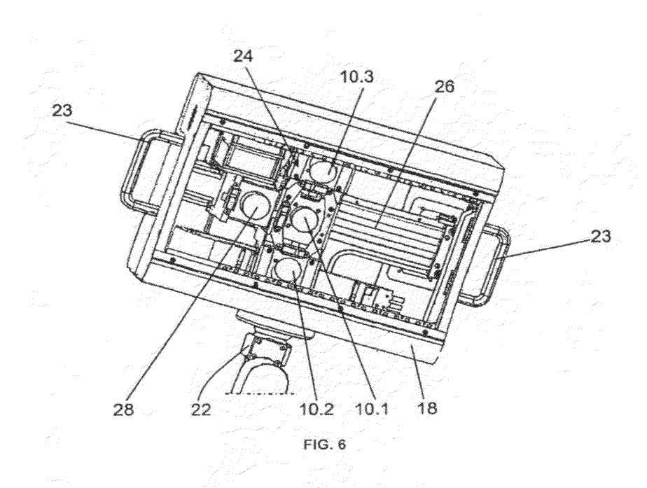

FIG. 3 is a schematic illustration of the arrangement comprising three light sources 10.1, 10.2, 10.3 and/or laser diodes and/or laser units for spatial irradiation along a direction of movement indicated by the arrow 16, with the various sub-areas A to D being irradiated sequentially. The light sources 10.1, 10.2, 10.3 are disposed on a laser system 24 (FIG. 6), which can be displaced on a frame, which is not shown here, including the associated guide element, in the direction of the arrow 16 and can be positioned in association with the various sub-areas A to D. The two lateral light sources 10.2, 10.3 or laser diodes are disposed pivotably with respect to a central light source 10.1 so as to be able to advantageously adapt to the contour of the surface to be treated. For the sake of simplicity, the light sources are referred to hereinafter as laser diodes, without, however, thereby limiting the invention.

The apparatus is preferably divided into the following five primary modules, the prefixed alphanumerical designations of which are used hereinafter.
M1 irradiation unit 2
M2 positioning element/positioning arm/articulated arm 6
M3 power supply unit including safety elements 4
M4 equipment cart 8
(M5 therapy set).

The M1 module—irradiation unit 2—is expediently divided into three sub-modules:
M1A laser unit 10
M1B control module
M1C housing 18 and irradiation unit 2.

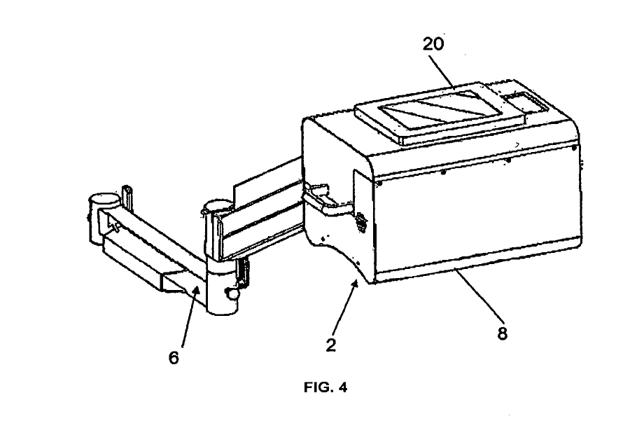

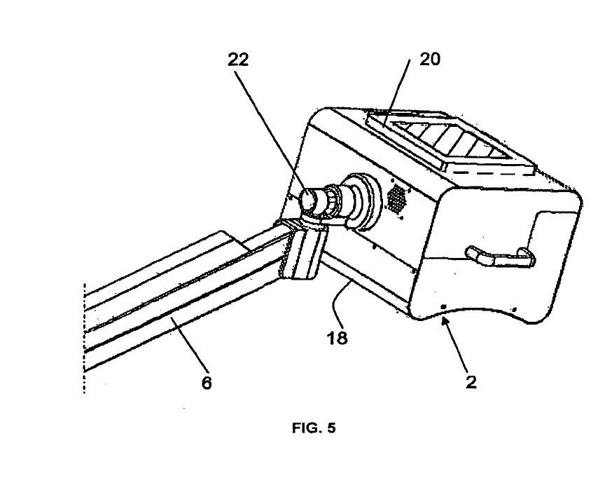

FIGS. 4 to 6 show views of a specific embodiment of the irradiation unit 2 comprising a housing 18 and a display 20 integrated thereon or therein. The display 20 is notably part of an operating unit, which will be described below. The, irradiation unit 2 is coupled to the articulated arm 6 by way of a ball joint 22 of the positioning element for the purpose of predefinable and/or free positioning with respect to the therapy area, wherein the user expediently grasps the handles 23 on the housing. The laser system 24 contains the three laser diodes 10.1, 10.2 and 10.3 and can be displaced and/or positioned by means of a linear guide 26. Moreover, a camera 28 is provided, which is preferably coupled to the laser unit 24 and is positionable. The travel of the laser system 24 is defined by the linear guide 26 and in this embodiment preferably is essentially 15 cm. Within the scope of the invention, the linear guide 26 may also be defined for a different amount of travel. The laser system 24 contains the three light sources and/or laser units 10.1, 10.2 and 10.3 in a spatial or 3D arrangement.

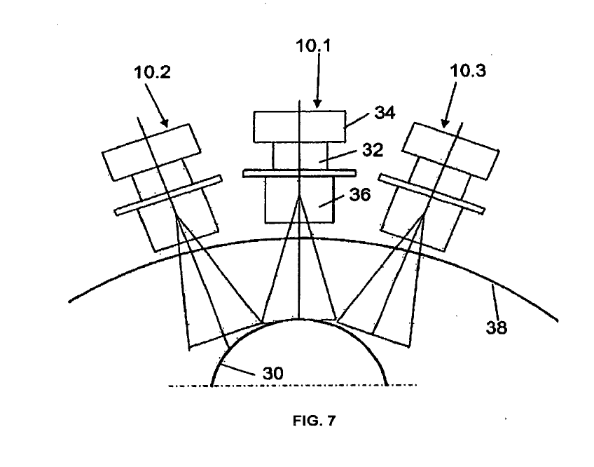

FIG. 7 shows the arrangement of the three laser units 10.1 to 10.3, wherein the lateral laser units are disposed pivoted with respect to the central laser unit 10.1 substantially by 20° and wherein the rotation center is located in the target region or the therapy area. By way of example, the line 30 indicates the surface of a thick leg that is irradiated by means of the laser units, the diameter of the leg being 105 mm. Each of the three identical laser units, which are installed in the irradiation unit 2, contains a laser diode 32, a heat sink 34 and a lens 36. The line 38 indicates a translucent and/or transparent protective pane of the irradiation device, wherein this protective pane is notably made of polycarbonate. Each of the laser units substantially comprises
a laser diode having internal or external cooling
a collimation lens
two microlens arrays for homogenizing the beam
lens mounts and holders.

The characteristics and/or functions and/or properties of the modules and components, some of which are formulated hereafter as requirements, and the cooperation of the same are implemented individually or in obvious combinations in the apparatus according to the invention and/or are realized with the method according to the invention.

M1A Laser Unit

The laser unit 10 notably meets the following requirements:
use of laser as the light source, wherein the laser light has a wavelength of preferably at least approximately 661 nm (+/−20 nm)
the wavelength of the light must correspond to that which activates the dye that is used
power density of at least 10 mW/cm² on the irradiated wound surface so as to activate the dye
required energy applied per unit area to the wound surface or effective dose: 3-5 J/cm²
The distance of the irradiation unit is adjusted to the irradiation system so as to ensure optimal irradiation (correct power density and the like). The control distance is 10 cm (laser diode outlet point to the wound surface) so as to be able to record a camera image and attain irradiation at the proper power density. The minimum distance is 8 cm to prevent contact with the wound by the device (especially with curved surfaces). The maximum distance is 12 cm to ensure sufficient power density (measurement at the respective center of the beam).
The housing 18 being designed such that no contact occurs with the patient at the specified distance of the laser unit.
One-time irradiation irradiates a wound area that covers the majority of the wounds. A definitive size cannot be established because the shapes of these wounds vary greatly. However, the objective is to be able to irradiate a wound having a size that corresponds approximately to half the surface of a human lower leg—ideally within 10 minutes. During a treatment (without repositioning the irradiation unit), it must be possible to irradiate a flat area measuring 15×13 cm (or 15×10 cm when irradiating a curved area).
This minimum wound area to be irradiated refers to the clinically effective region in which the stated 100 mW/cm² power density exists. Attention must be paid to the Gaussian distribution. If needed, appropriate lenses or beam homogenizers are to be used. Homogeneous irradiation of the entire area; the power density should be no less than 100 mW/cm² in any region; the power density described above may be exceeded in areas where the beam cones overlap.
The irradiation duration should not exceed 15 minutes. The maximum treatment duration is 15 minutes; this duration may be exceeded for very large areas; the region that can be treated within 15 minutes is predetermined.
The irradiation should take all three dimensions into account. Any reduction due to potential beam angles must be considered, and measures should be employed to prevent irradiation errors (insufficient power density→insufficient energy). The irradiation is preferably carried out from the three spatial directions using a substantially uniform power density. When irradiating curved surfaces (such as a leg), the power density must not drop below the 100 mW/cm² in sites that are further removed from the light source. The following limits must not be exceeded in sites located more closely:
500 mW/cm² (limit for laser class 3B)
±20% of the rated output power (according to EN 60825)
The consistency of the light power that is output is assured over the entire irradiation duration (=>power fluctuations, failure of individual light sources . . . ), wherein distance control and/or constant control of the output power, in particular, are carried out.
Appropriate dissipation of the waste heat generated by the light sources is assured. Due to the increased number of light sources required for irradiating the area, increased temperatures are to be expected in the region of the light sources. Appropriately designed cooling for the light sources is a prerequisite. Cooling is to be designed for uninterrupted operation. Relevant standards according to the maximum permissible surface temperatures are to be observed. The heating of the light source or device must not exceed the maximum temperature values of the light sources or standard specifications. Heating of the outside surfaces: according to standard 60601-1:1990: maximum surface temperature for application parts 41° C.=parts that come in direct contact with the patient. The maximum surface temperature for metallic parts that are held constantly by the user is 55° C. Impermissible draft in the region of the wound (caused by fans) is prevented.
A calibration apparatus is provided for reliably and easily measuring and adjusting the output radiation.
Impact-/shock-proof mounting of the laser diodes so as to prevent damage by striking against the device and the like. The impact is cushioned by the elastic mounting of the laser diode carrier or the entire irradiation head mechanism.
The output laser power is fixed and is adjusted in accordance with the respective power density and the fixedly defined distance. The output power is constantly controlled to a fixedly defined value, in particular control electronics (laser diode driver module).

Interfaces Between M1A and Other Modules:
M1B: laser diodes are activated or deactivated by M1B.
M1B: attachment of the laser diodes (including cooling and any lenses) to the moving part (“carriage”) of the linear guide. A laser diode carrier is designed as a frame, which forms the moving part of the linear guide.
M1B: installation of the laser diode driver modules
M1C: ventilation outlets in the housing
M2/M3: power supply for the diodes

M1B Control Module:

The control module comprises:
A linear drive for linearly moving and positioning the laser unit (for example using a stepper motor and incremental encoder for position determination). The linear mobility of the laser unit is defined along the primary extension direction of the irradiation unit. The length of the linear unit is in accordance with the length of the maximum area that can be irradiated, preferably in the range of 10 to 25 cm, and more particularly in the range of 14 to 20 cm.
A respective drive unit for positioning the lateral pivotable laser diodes (for example using a stepper motor and incremental encoder for position determination). It is disposed toward the central laser diode so as to be able to irradiate both curved areas (such as legs) and flat areas (such as backs). The laterally disposed diodes can be pivoted in a preferably defined angular range of 10° to 30°, and more particularly substantially 20°.
At least one camera, which supplies an image of the area that can be irradiated. Preferably a color camera is provided, which records and supplies an image of the total area that can be irradiated. If the camera angle is not sufficient at the defined distance from the wound surface, optionally several cameras or a movable camera (dedicated linear guide or movement jointly with the laser unit) that scans the area may be used. It should be noted that, if the wound surface is curved, the camera angle for the image will not correspond to the beam direction of the outer laser diodes. It is further preferable for at least one additional light source to be provided so as to obtain a good camera image.

Distance Sensors:
A distance sensor in the region of the central laser diode, which moves together with this diode.
One at a long end (optionally both ends) of the irradiation unit, which measures the distance of the irradiation unit/laser diodes from the wound.
A respective distance sensor, which measures the distance of the outer pivotable diodes from the wound surface. The distance sensors are provided for central and lateral laser diodes and are fixed at one end of the irradiation unit. A preferred fail-safe variant contains two distance sensors on at least the central laser diode. The distance sensor is, or the distance sensors are, designed in particular as ultrasound or laser sensors. The measuring accuracy preferably ranges around ±5 mm.

Display, Especially Comprising a Touch Screen:
Input is performed using a stylus or finger, wherein operation using disposable gloves should be possible. The orientation of the displays is defined so that a user standing at the side of the equipment cart can recognize or interpret them easily and/or well. The display (at least 8-inch) is used to depict the camera image, superimpose it with a grid, depict the selected fields or depict red/green bars to assist the spaced positioning. The display is disposed so that even users who are not very tall are able to read the display in any position.

Input Keys:
scan (start camera recording)

Start-stop (start/stop irradiation). Preferably a membrane keyboard comprising all the necessary input keys is provided. Moreover, the input keys are optionally integrated in the display. Additionally, the input keys are designed and/or disposed so that a keystroke does not alter the adjusted position of the irradiation unit.

Emergency-Off Push Buttons:
emergency-off push buttons on the irradiation unit and equipment cart, upon the actuation of which all drive motors and the laser radiation are stopped.

Control Software:

The control software, for example in the form of an embedded systems, fulfills and/or controls the following functions:
processor and electronic components:
processor, on which the control software runs and electronics package for controlling drives, sensors, camera, display, input keys and emergency-off. Moreover, protection of the functions of the control software/electronics is provided for so as to activate the emergency-off function in the event of a system failure (watchdog hardware/software).
access guards on the lower face to protect individuals when “reaching into” the device: the pause mode is activated (laser off) when an object enters a definable distance from the laser beam outlet opening.
mechanical components:

A frame, which bears all the components, in particular linear guide, drives, camera, display, operating elements, sensors, or access protection for diodes.
laser operation indicator according to relevant standards, such as:

green: laser is ready (key-operated switch is actuated, device is switched on, and temperature of the laser diode is in the correct range).

yellow/orange: for laser emission—start was pressed—device emits light radiation

red: error (not absolutely necessary)

The indicators either integrated in the display according to relevant standards (EN60825, EN60601-1-22) or designed as dedicated lights (such as LED, LED strips etc.).
manual reactivation unit:

Irradiation must not continue automatically after power failure or the like.
storage of operating times:

The total operating time of the device and the laser operating time of the individual diodes are permanently stored.

Interfaces Between M1B and Other Modules:
M1A: activating/deactivating the laser diodes and various monitoring functions by direct actuation/sampling of the laser diode controller (voltage-controlled);
actuation/monitoring for all three laser diodes.
diodes on/off, voltage-controlled. Monitoring of the laser diode temperature by way of a voltage signal from the laser diode controller. Irradiation is only approved when the temperature of the LD is in the established range. Monitoring of the laser diode system, warning in the event of variance from the permitted range (advice with respect to damage). Monitoring of the rotational speed of the laser diode fan coolers (alarm if cooler is not running). Optional: control of the laser power by voltage signal.
M1A: attachment of the (three) laser diodes (including cooling and any lenses) to the moving part (“carriage”) of the linear guide. Frame accommodating the diodes is part of the linear guide. Interfaces to the laser diode unit notably include rubber jaws/rubber rings and the like.
M1A: installation of the laser diode driver modules:

Space is provided within the irradiation unit for installing the (three) laser diode driver modules, including heat sink/fan (approximate dimensions 120×70×60 mm; installation stationary or on the “laser carriage”).
M1C: connection of the frame to the housing and installation of the display, of the input keys/membrane keyboard, or emergency-off in the housing. A rotary movement, in particular about a horizontal axis along the transverse irradiation unit axis, is preferably allowed in the region of the connection between the articulated arm and irradiation unit, wherein the irradiation unit can preferably be automatically locked in the selected position.
M2/M3: power supply
M2: attachment of the irradiation unit to positioning element/positioning arm.

M1C Irradiation Unit Housing:
housing comprising fitted operating elements of the user interface and comprising ventilation outlets: air inlets and outlets on the side (or located at the top, however protected from direct penetration by liquid if possible), possibly comprising dedicated fan so as to allow a slight constant draft that transports the waste heat of the laser diodes to the outside. Installation of the operating elements preferably on the upper face of the housing. The lower face of the housing is generally open because this is where the laser radiation exits.

Optional: dedicated touch screen (independent of the irradiation unit) comprising dedicated attachment (articulated arm) to the equipment cart.
The lower device face should be variably closable, which is to say only the respective laser outlet point is open (or covered by a glass/plastic panel), while the remaining opening is closed by a kind of “curtain” or the like. Rigid covering of the lower face of the irradiation unit as protection against access and dust/dirt: transparent polycarbonate (PC) panel, curved (r=240 mm) along the longitudinal device axis, 2 mm thick.
handles for manually positioning the irradiation unit over the wound area. At least two handles disposed laterally on the housing.
monitoring of the housing leakage current:

AKM1C4: housing leakage current is within the boundary values (for example by supplying the irradiation unit with low voltage and applying all relevant sections of EN60601).
housing shape should be adapted to the body shape to prevent contact with the wound surface. The housing must be designed such that no contact occurs with the patient at the specified distance of the laser unit, notably with curved treatment areas.
EMC shielding of the electronics by housing (material)
protection against outside moisture:
the housing should offer protection from penetrating liquid (in particular from above) into the electronics beneath (gaskets on the fitted operating elements and the like).
use of suitable materials for the housing, notably anodized or powder-coated aluminum.

M2 Positioning Element Module:
configuration in particular with articulated arm 6 and/or articulated stand, which are ideally equipped with a quick central fixation. Articulated arm/articulated stand with quick central fixation, 100% fixed positioning during the entire treatment duration; locked in transport position. For the connection to the irradiation unit 2, the positioning element preferably contains the aforementioned ball joint 22, wherein this joint is also expediently equipped with a quick central fixation and/or fixed positioning.
The irradiation unit should be able to irradiate any part of the body (with the patient lying down, with this applied in the height range of common patient beds). Horizontal range from the outer edge of the equipment cart to the center of the irradiation unit: at least 100 cm. Vertical range (beam output plane from 63 to 136 cm high).
Cables for the power supply from the power supply unit to the irradiation unit. Cable should be run hidden, is so far as is possible, in or along the articulated arm.

M3 Power Supply Unit Module Including Processor and Safety Elements:
housing comprising installed central on/off switch.
power supply with mains connection line. Transformation of the mains voltage to low voltage, which is forwarded to the irradiation unit by cables.
EMC compatibility of the power supply or shielding by the housing in compliance with EMC regulations.
mains unit or transformer:
voltage 5V (laser diodes, real-time system) and 12V (PC system/processor)
power consumption of laser diodes approximately 6 A
processor, which advantageously is designed as a PC system and, if needed, is arranged separately and connected via a cable.

M5 Therapy Set Module:

The component referred to as the therapy set comprises the materials required for each individual aPDT application:
photosensitizer: given the availability of HELBO Blue Kutan, which has been fully developed and is already approved for application to skin surfaces, this dye solution will be used for use with this device. The following parameters should therefore be regarded as predefined—wavelength near 661 nm, the absorption maximum of the photoactive component.
The photosensitizer fill quantity per therapy set should be sufficient to dye the maximum wound area that can be irradiated. 0.5 ml HELBO Blue Kutan is sufficient to dye approximately 50 cm².
optional/future variants:
therapy sets comprising variably large fill quantities for varying wound sizes.
The further content of the therapy set should include all aids that are required for the application, dabbing, rinsing the wound and the like.
All individual parts of the therapy set are to be placed into circulation in a sterile manner in suitable packaging.

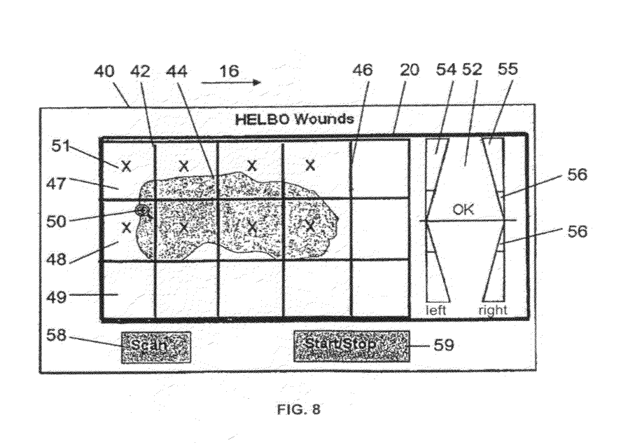

FIG. 8 shows a view of the operating unit 40, which is preferably disposed on the housing of the irradiation unit. The operating unit 40 contains the display 20, on which the live-camera image 42 of the wound area recorded by the camera and processed by the processor is inserted and the wound area 44 is depicted. Moreover, a grid field 46, notably generated by means of a processor, is inserted and superimposed with the live-camera image 42. Each of the preferably square grid fields corresponds to an irradiation field generated by means of the laser unit. This is based on the laser system comprising three laser diodes described based on FIG. 6, by means of which three irradiation fields 47, 48, 49 can be generated simultaneously. The irradiation fields 47, 48, 49 are located next to each other and transverse to the direction of movement of the laser system, as indicated by the arrow 16. By way of the guide element and the drive unit, the laser system is displaced sequentially in the direction of movement 16 and positioned, according to the invention, such that the irradiation fields located next to each other in the direction of movement 16 can be irradiated consecutively such that matrix-like irradiation in accordance with the entire grid field 46 is carried out. So as to irradiate only the grid fields that cover the wound area 44, these are marked using suitable marking means, which are indicated here by crosses X. If the display 20 is designed as a touch screen, the marking is carried out by tapping or touching the aforementioned fields. As an alternative, the marking can be carried out, for example, by means of the processor or the PC system by a mouse click 50 on the respective field. In keeping with the selection that was made, only the accordingly marked fields of the wound area 44 are irradiated when the irradiation is carried out.

The display 20 further comprises a distance indicator 52, which indicates the positioning of the irradiation unit with respect to the wound area 44. If the bars or triangular symbols 54, 55 are all red, for example, the irradiation unit is located too far away at both ends. If, in contrast, the bars or symbols 54, 55 are green, for example, at least in the region of the tips 56, both on the left and right, the irradiation unit is positioned at the correct distance on both sides. The operating unit 14 further contains a scan key 58 and a start/stop key 59.

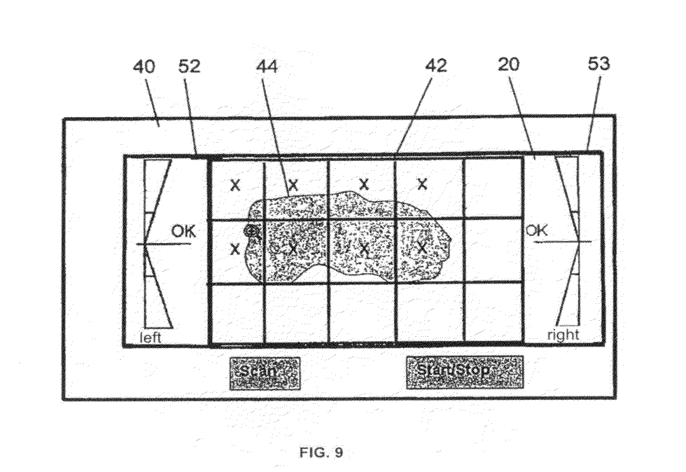

FIG. 9 shows an alternative embodiment of the operating unit 40 or of the user interface of the display 20, wherein the distance indicator is divided into two parts 52, 53, which are disposed to the left or right next to the camera image 42. This achieves an improved intuitive arrangement in accordance with the spatial or geometric relationship of the operating unit to the wound area.

The operating principle of the apparatus and the various steps of the method will be described in more detail hereafter.

Step 1: Positioning the Irradiation Unit Over the Wound

A Irradiating “long wounds”, wherein the length of the wound in the direction of the longitudinal axis of the irradiation unit and/or the direction of movement is larger than two irradiation areas: The person performing the treatment positions the irradiation unit 20 parallel over the wound area using the aforementioned handles. The two distance sensors are positioned at the ends of the irradiation unit 2, respectively. One distance sensor is preferably disposed on the laser system at the central diode, which is initially positioned at one end of the irradiation unit 2. The second distance sensor is rigidly disposed at the other end of the irradiation unit 2. The two distance sensors measure the respective distance of the plane of movement of the central laser diode from the underlying wound surface or wound area.

B Irradiating short wounds, the length of which in the direction of the longitudinal axis is no greater than two selectable irradiation areas. Contrary to variant A, during the adjustment, the distance is measured only at one end of the irradiation unit 2, preferably by means of the distance sensor of the central laser diode. When this distance is correct at one end, the wound can be scanned. Thereafter, it is only possible to select an irradiation area if the distance is correct. It should be noted that during scanning both the camera image and the distance are determined.

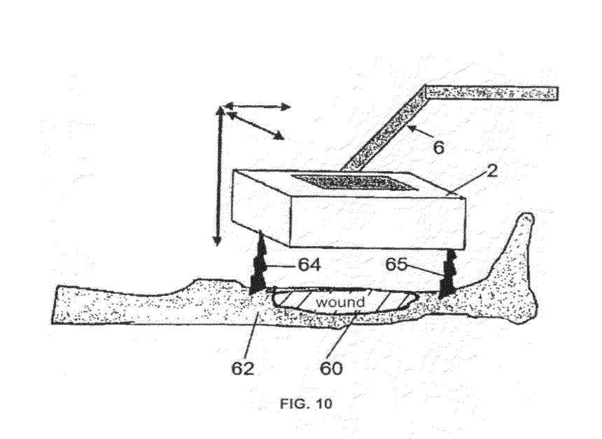

FIG. 10 shows the positioning of the irradiation unit 2 over a wound 60 on a lower leg 62. The irradiation unit 2, which is attached to, or articulated to, the positioning element 6, is positioned by the person performing the treatment parallel over the wound area 60 in accordance with the aforementioned variant A, wherein the irradiation unit is located substantially parallel to the longitudinal axis of the wound. The arrows 64, 65 indicate the measuring beams of the distance sensors. Using the distance indicators described based on FIG. 8 or FIG. 9, the distance is graphically depicted on the display, notably in color, using a symbol or a bar for each of the two distance sensors. For example, green bars are illuminated if the distance is within the correct defined range, otherwise red bars are illuminated. The following meanings may apply, for example:
dark green: the distance from the defined ideal distance, for example 100 mm, is +/−5 mn and is substantially adhered to.
light green: the distance from the ideal distance is +/−10 mm; small tolerance.
bright red: the distance from the ideal distance is +/−20 mm; increased tolerance.
dark red: the distance from the ideal distance is greater than 20 mm; excessive or impermissible distance.

Irradiation, more specifically by activation of the start/stop key, may only be approved if the correct distance, in particular the ideal distance depicted in green, has been set and the operating temperature of the laser diodes is also in the defined range.

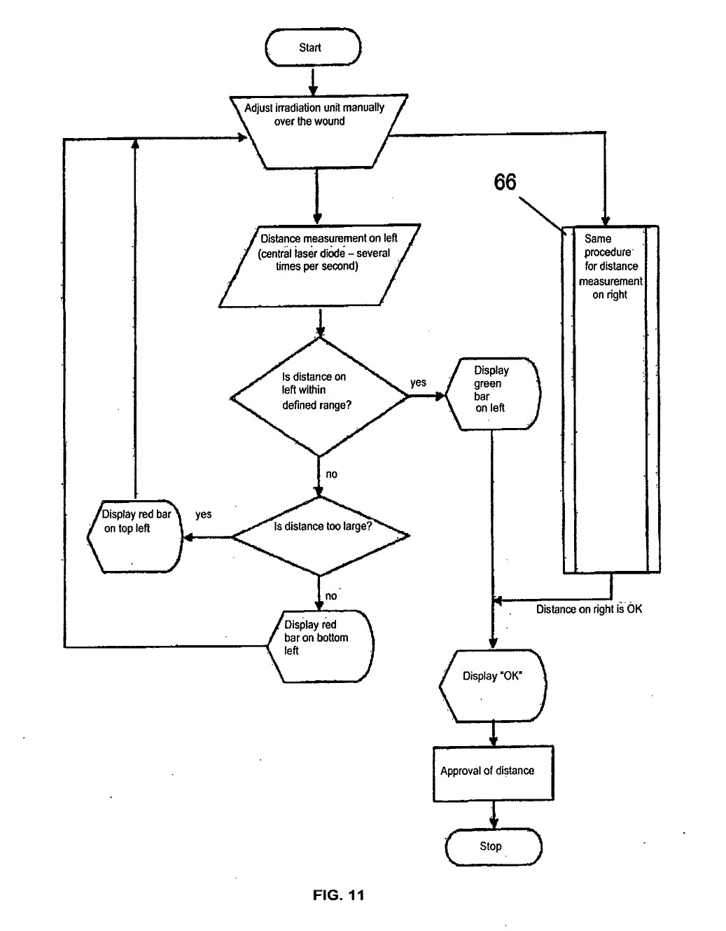

FIG. 11 shows the relevant flow chart for positioning the irradiation unit. The flow chart is provided in its entirety for the left distance sensor, while the equivalent distance measurement by means of the right distance sensor is indicated by the block 66.

Step 2: Orienting the Outer Pivotable Laser Diodes

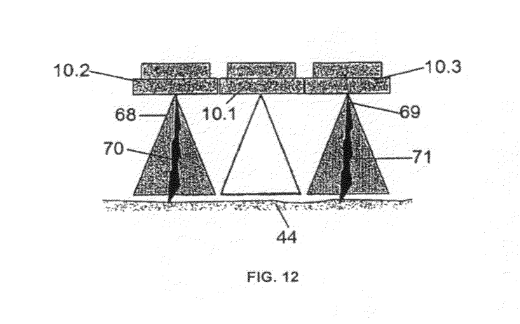

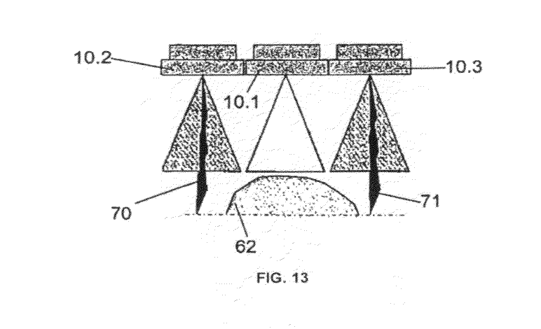

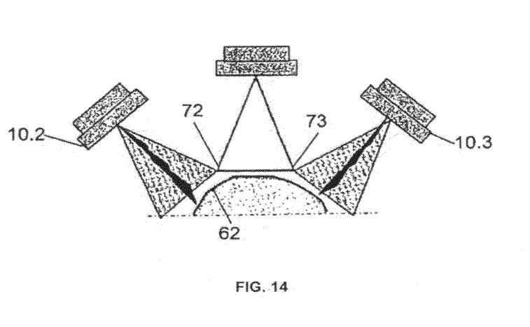

This method step is carried out for the embodiment of the apparatus described in particular based on FIGS. 3, 6 and 7 and will be described in more detail based on FIGS. 12, 13 and 14. The distance of the central laser diode 10.1 has already been correctly adjusted according to step 1. A respective distance sensor 68, 69 at the outer diodes 10.2 or 10.3 measures the distance from the wound surface. According to FIG. 12, planar irradiation is applied to a substantially planar wound area 44, with no adaptation of the two outer diodes 10.2 and 10.3 being required. The arrows 70, 71 indicate the measuring radiation of the distance sensors, while the laser radiation is indicated in triangular shape. FIGS. 13 and 14 show spatial irradiation of a lower leg 62. The displacement direction of the laser diodes 10.1, 10.2 and 10.3 is perpendicular to the drawing plane towards the back, or into the figure. FIG. 13 shows the starting position, with the distance of the central diode 10.1 already being adjusted. The two lateral diodes 10.2, 10.3 still have to be adapted to the leg curvature because the distance sensors initially supply values that are too large. As shown in FIG. 14, the two outer or lateral diodes are automatically pivoted inward, by means of associated drives, until the correct distance has been reached. The respective rotation center is preferably the contact point 72, 73 of the beam rays shown as triangles. It is of particular significance that the orientation and/or distance measurement of the lateral diodes 10.2, 10.3 is carried out continuously, and more specifically preferably several times a second. Movements by the patient are thus preferably responded to and/or the distance is adapted to changing anatomic circumstances during displacement of the laser unit.

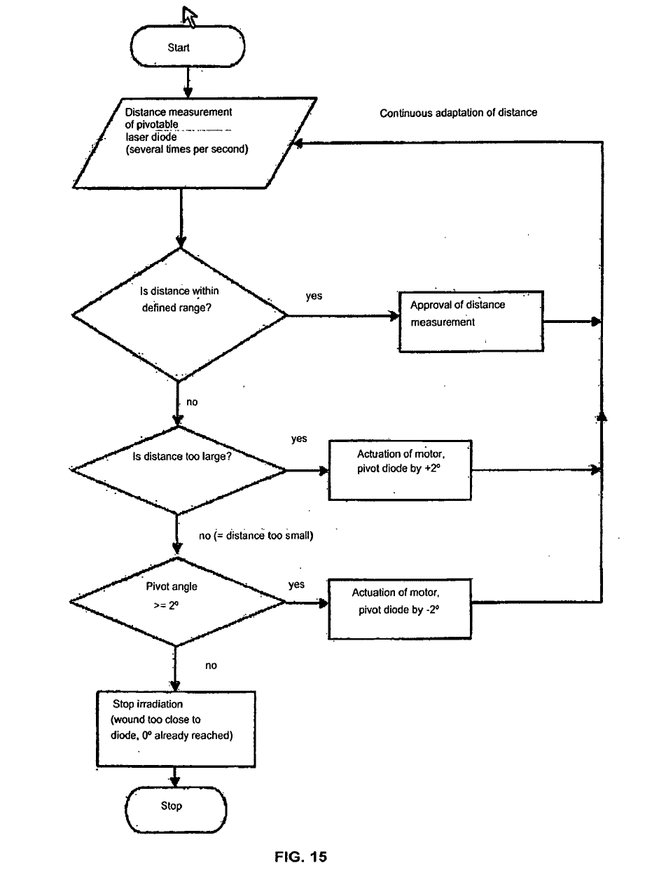

FIG. 15 shows the flow chart for positioning the two pivotable lateral diodes.

Step 3: Imaging the Wound Area

After the irradiation unit is positioned, the camera integrated in the irradiation unit records an image of the wound, the size of which corresponds at least approximately and/or precisely to the area that may be irradiated. As an alternative, the camera image may be composed of several individual images, wherein preferably several cameras are provided, or a movable camera is provided. As described already in accordance with FIG. 8, the image of the maximum wound area that can be irradiated, which has been recorded with the camera or cameras, is depicted on the display and overlaid with a grid, which depicts and/or corresponds to various irradiation positions.

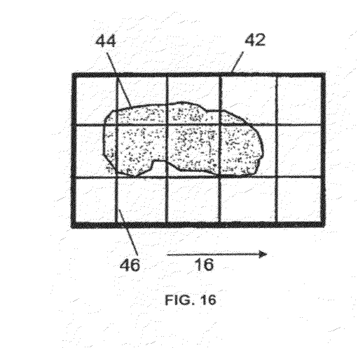

FIG. 16 shows the camera image 42 comprising the wound area 44 and the inserted grid 46 on the display. The grid is divided into three sections in a direction transverse to the direction of movement 16, wherein each grid field in this direction corresponds to the respective irradiation position of the three laser diodes. A subdivision in five positions is provided, for example, along the direction of movement 16.

Step 4: Selecting the Irradiation Area

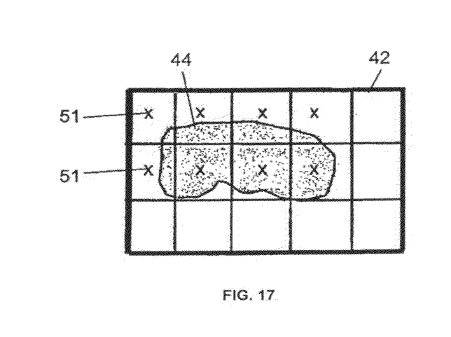

Based on step 3, the grid fields which are to be irradiated are defined, as depicted in FIG. 17. Initially, no field is selected, and this is selected by selecting the respective field. If the display is designed as a touch screen or touch panel, finger pressure on the appropriate grid field alters the selection of the field. Selected fields are preferably marked and/or depicted in a visually differing manner, for example shaded or hatched, wherein the recorded original camera image still must be recognizable. By way of example, the markings 51 are depicted here by crosses in accordance with FIG. 8.

Step 5: Starting the Irradiation

The irradiation is started by pressing the start/stop key 59 shown in FIG. 8. It is of particular significance that the start/stop key is only active when the distance has been selected correctly, approval being given in accordance with the distance measurement and, moreover, with at least one field selected. The laser unit begins the irradiation at a starting point, which is located at one of the two end points of the guide element, notably the linear guide. Each position is irradiated for a defined period in accordance with the positioning of the laser unit in the direction of movement or displacement direction of the linear guide, and more specifically after the power density is reached depending on the design of the laser unit. Thereafter, the laser unit or the laser system is moved to the next position, where the irradiation is continued. It should be pointed out that a selection field does not necessarily have to correspond to an irradiation position. The size of the region or area irradiated at one time by a laser diode in the direction of the displacement direction may optionally be smaller than the opening of the grid field or grid fields. In such a case, two or more irradiation positions are required to irradiate a selected field.

For example, if the irradiation duration per field is 40 seconds, a selected field corresponds to five irradiation positions of, for example, 6 mm. According to the invention, only the fields that have been selected are irradiated. The diode is deactivated for a respective position if the position is not selected. If no field has been selected at a position in the displacement direction, or if none of the three fields that are present next to each other with respect to the travel direction have been selected, the laser unit is immediately moved to the next positions.

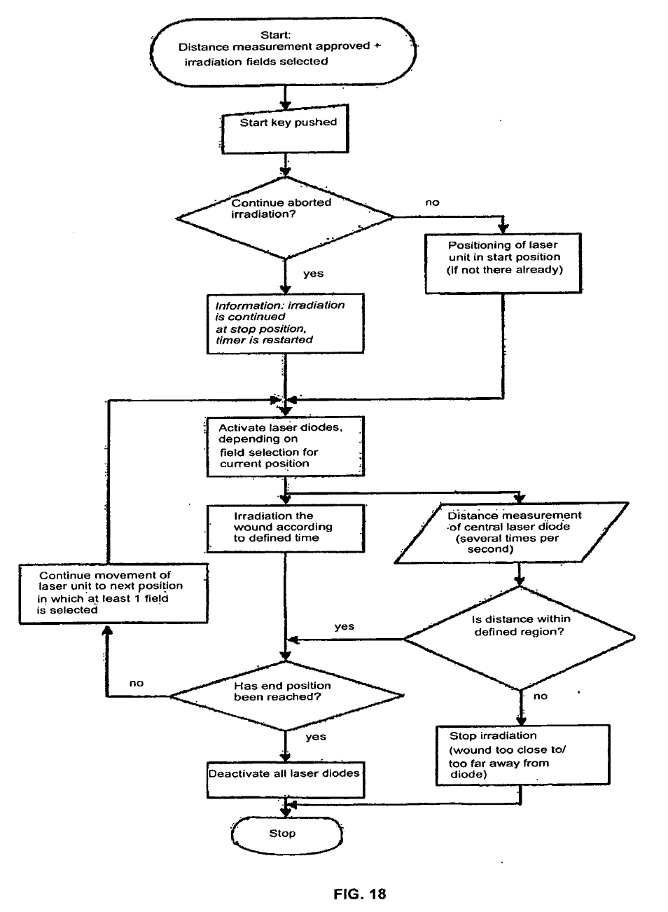

FIG. 18 shows a flow chart for irradiation, which more particularly includes the continuous distance control. The distance measurement and the adaptation of the lateral laser diodes are carried out, in parallel or simultaneously, as shown in FIG. 15.

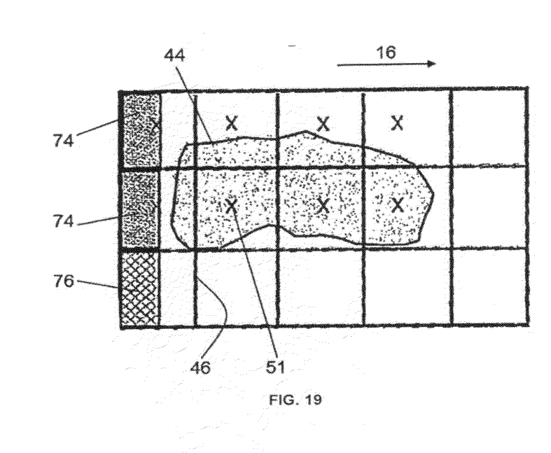

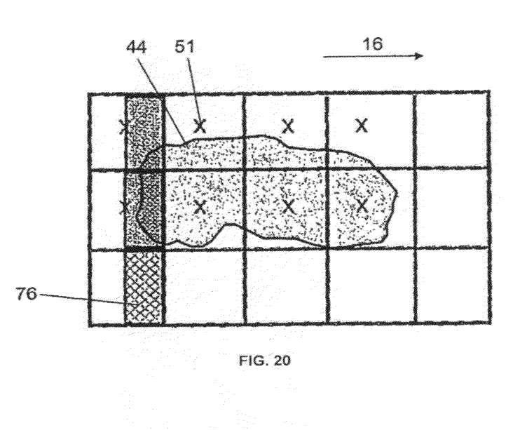

FIGS. 19 and 20 show camera images of the wound or wound area 44 along with the grid 46 and the fields selected in accordance with the markings 51. The areas 74 highlighted in gray depict the areas located underneath that are physically irradiated at the respective time by the laser diodes, wherein the associated laser diodes are activated. In contrast, the areas 76 highlighted with crossed hatching are not irradiated, because the associated laser diode is deactivated. FIG. 19 shows a first irradiation position of the laser system and FIG. 20 shows a second irradiation position of the laser system in the direction of movement 16, more particularly each in the same grid fields 46.

Step 6: Continuous Distance Control

So as to prevent distance changes resulting from movements by the patient or the like during the therapy, according to the invention the distance between the central laser diode and the wound surface is continuously controlled. The distance of the central laser diode is preferably continuously monitored during irradiation of the target area and/or during the linear further movement of the laser unit. If the distance is outside the defined range, the treatment or irradiation is interrupted. As soon as the required distance has been re-established, the therapy can continue following an interruption, in particular by pressing the start/stop key, more specifically starting from the current position.

Step 7: End of Therapy

Irradiation is terminated when

a) all fields have been irradiated;

b) the therapy was automatically interrupted due to an incorrect distance and was not continued;

c) a maximum total therapy time is reached. This way, the light sources are automatically shut off in the event of an error;

d) the start/stop key is pressed after therapy start and before the end of the therapy; and

e) the emergency-off push button is pressed.

The linear movement of the laser unit is automatically deactivated after all selected fields have been irradiated, wherein preferably audio and/or optical feedback is provided. So as to prevent renewed start-up of irradiation of the wound after the end of therapy by simple and/or inadvertent pressing of the start/stop key, all areas to be treated are deselected after the therapy has ended. Moreover, the start/stop key advantageously has a type of pause function. As long as the predefined distances are maintained, the therapy can be resumed after a stop, or even after an automatic stop resulting from an incorrect distance, by pressing the start/stop key again; the renewed start occurs at the position irradiated last, wherein the irradiation time for this position is restarted. The irradiation time can optionally also be defined or calculated for the renewed start of the aforementioned position, in particular from the remaining time plus a buffer period, which is preferably predefined at least approximately at 5 seconds.

The characteristics, properties or operating principles provided hereafter as requirements and criteria for the various components or elements of the apparatus are implemented within the scope of the invention in addition, or as an alternative, to the description provided above, and more specifically individually, or in advantageous combination, in the apparatus according to the invention, or carried out by the method according to the invention.

Drive

It should be possible to irradiate an area measuring approximately 15×13 cm using three laser diodes. For this purpose, it is required that the diodes be moved by a linear drive. The linear drive is actuated by the real-time system.

The two outer diodes are disposed at a fixed angle of 20° (with respect to the irradiation plane or central diode).

Color Camera

The camera picture depicted should represent an image of the entire area to be irradiated. If it is not possible to depict the area using one image, it is possible to use several cameras or one camera having a linear guide (dedicated or with laser guidance). The camera image that is recorded must be processed by the software of the PC system prior to depiction on the display so as to yield the most authentic depiction of the wound possible (optical control of the recorded image).

Additional Light Source

If the lighting conditions are insufficient because of the small distance from the wound, a light source is to be provided. The light source is to be activated for the duration of the images (scan). When the start screen is active, the light source is only activated when one of the distance sensors measures a distance of <200 mm (so that the light is only on when the device is used, and not immediately when it is switched on). The light is switched off during irradiation.

Distance Sensors

The measurement of the distance between the skin and the laser diode should amount to 100 mm (AKM1A4). The measuring accuracy must be at least ±5 mm. It should be possible to change the distance value in the Helbo service menu. Suitable sensors (infrared) must be used for measurement. One distance sensor is to be used for each laser diode, the sensor measuring the distance to the center of the irradiated area (relative to the longitudinal axis of the device). In addition, a sensor is required on the housing. The device is adjusted by means of the sensor on the housing and the sensor on the central laser diode (monitoring and comparison of the measurement result to the actual distance).

Display

A touch screen measuring at least 8″ must be used to visualize the camera images, to select the areas to be irradiated and for operation.

Operation must be possible using disposable gloves. The display and the housing must be protected from moisture penetrating from above (optical control of the camera image, functional check of the touch screen).

Input Keys

The key-operated switch interrupts the entire power supply of the real-time system and PC system. After being activated by the key-operated switch, the real-time and PC systems start up and the start screen is displayed. This also ensures that irradiation does not automatically start following a power failure. When the device is switched on, the display is switched off when a stand-by key is pressed, and depending on the technical feasibility and usefulness, the PC and real-time systems (=stand-by mode) are also shut off. This mode is indicated by an illuminated red LED (perhaps directly on the push button). Activation from stand-by: by again pressing the stand-by key (if technically possible, also by tapping the touch screen)—all system components are powered up again—the start screen is displayed. A green LED indicates operational readiness (the green LED can be used for this)—the red key LED is then deactivated (or is green).

The stand-by key is not effective during irradiation or in the pause mode.

After 20 minutes of inactivity (the time configurable), the stand-by mode is automatically activated.

The following operating elements are provided on the display and/or touch screen:
continue/back:
switch between screens
scan:

starts the recording of the camera image
start/pause:

starts or interrupts the irradiation of the selected fields
abort:

interrupts the therapy
deactivation of alarm signal (“audio pause”)

Only those operating elements that are allowed to be activated at that time, depending on the therapy flow, can be selected. Operating elements that can be activated are to be highlighted graphically or in color as compared to the non-active elements (such as by a border).

All operating elements are to be protected from penetration by liquids.

(Confirm the keys and perform functional test).

Emergency Off

The emergency-off push button stops the real-time system (including all drive motors and laser diodes). The emergency-off push button must be designed in accordance with guidelines (EN 60601-1-22).

The remote controllable safety element is treated like an emergency-off push button. If emergency off is actuated, power supply is interrupted to the following components:
linear drives
laser diodes
real-time system

Because of the high power consumption of the laser diodes, the power supply is interrupted by a relay.

A message is output on the screen indicating that an emergency off was triggered: “Emergency off or external safety circuit was activated. Please check the cause of the interruption and then restart the therapy.”

After the emergency-off push button has been deactivated, the system is re-initialized, the message disappears (without actuation by the user) and the start screen is displayed. (Actuate emergency off and door contact switch).

Access Guard

An access guard is to be provided on the lower face of the device. The laser radiation must be interrupted (pause mode) if anyone reaches into the device during irradiation.

The access guard is implemented by the distance sensors. If a distance from an object of less than 80 mm is measured during the continuous measurement (distance from laser diode outlet opening to skin, configurable), all drive motors and laser diodes are stopped. It should be possible to change this distance value in the Helbo service menu. A corresponding warning is shown on the display and the program is switched to pause mode.

Laser Operation Indicator

The laser operation indicator must comply with the standards EN 60825 and EN 60601-1-22 and can be provided either on the display or by use of LEDs. Three colors should be used for the operation indicator:
green:

laser is operational—key-operated switch is activated, PC and real-time systems are powered up, and laser diodes have the operating temperatures
yellow

laser emission
red:

An error has occurred:
laser diode is defective
other critical faults

Protective laser goggle notice: avoid red symbols and instructional text as much as possible! (Visual inspection of the operation indicator).

Storage of Operating Times

The operating times of the laser diodes must be documented.

It should be possible to reset the stored operating times. (Read operating times after treatment has expired).

Control Module

The “M1B” control module comprises the control and positioning of the laser diodes. The “M1B” control module is divided into two components:
real-time system:

All safety-critical processes, such as the actuation of the laser diodes, distance measurement, access guard and the like, are managed by this component. The control must be real-time capable.
PC system:

The PC system manages the processing of the image, the selection of the areas to be irradiated, storing the operating times and user interaction.

Interface for Embedded Components

The PC system and the real-time system communicate via a serial interface. Communication is assured by a transmission protocol. A “heart beat” is to be defined in the protocol and must be periodically transmitted. If one of the components does not respond, emergency deactivation must be initiated. An error message is shown on the display. (Interrupt communication of the PC system and real-time system, error must be displayed, and the treatment is aborted).

Laser Diode Current Monitoring

The power consumption of the laser diodes must be monitored during irradiation. If the variance in power consumption exceeds 20%, the treatment must be aborted because the diode may be defective.

The power is monitored by an output of the laser controller.

EMC, Explosion Protection

The EMC standards (EN 60601-1-2) must be taken into consideration when developing the hardware; the explosion protection provisions according to EN 60601-1 are also to be observed.

Remote Controllable Safety Element

Door contact switches according to standards EN 60825 and EN 60601-1-22.

If the switch is actuated (contact is opened), the laser diodes must be deactivated (function similar to emergency off). The electronic circuit for this function is to pass testing in accordance with EN 60601-1 (surge energy capacity).

Real-Time System

Tasks

The real-time system is in charge of
approving operational management
controlling the linear drive
measuring distances
controlling the laser diodes

Approval of Irradiation

After the irradiation unit is adjusted, the entire length of the irradiation element is traced once so as to measure the distance at each position (the minimum and maximum distances are determined for each selectable area, resulting in information as to whether the area is entirely within the valid range). The valid ranges (8-12 cm distance, it should be possible to change these distance values in the Helbo service menu) are transmitted to the PC system. If the distance is too small, the fields cannot be selected.

Distance Measurement

The distance must be measured continuously by the real-time system. The sensors for the distance measurement are connected to the real-time system. The distance of each sensor is read several times a second and processed.

Deactivation if Distance is too Small

If the distance is outside the permitted range <<80 mm or >120 mm, it should be possible to change the distance values in the Helbo service menu—a dedicated set of limits should be provided (not those from the scan)—emergency deactivation of the laser diodes must be performed. The program is interrupted and a message is sent to the PC system. (Reduce distance—deactivation must be performed).

Start of Irradiation

The irradiation process may not be started until the distance from the wound is in the green range of the area to be irradiated.

(Irradiation must not be started if the distance is too small—distance is changed after selection of the areas).

Irradiation

When starting the irradiation for an area, the laser unit must first be positioned. Then, the laser diode is activated for a certain duration (40 s). The duration is dependent on the power output by the laser diode and it should be possible to change it in the Helbo service menu.

When the time for the area to be irradiated has expired, the laser unit is positioned over the next area. The laser diodes should not be deactivated and reactivated during each step (if 2 areas are irradiated consecutively). Areas for which irradiation is not selected are omitted; if no further area exists, the irradiation is properly ended. An area corresponds to 5 irradiation positions in the longitudinal direction (it should be possible to change this value and the travel (6 mm) in the Helbo service menu).

The real-time system may only irradiate areas that were selected on the PC system. (Selection of the areas and control of the irradiated areas).

End of Therapy

When all areas have been irradiated, the laser diodes are deactivated, and the end of the therapy is reported to the PC system. (Wait for end of therapy).

Time-Exceeding

An area may be irradiated only for a certain period. The time is to be controlled by two systems that are independent of each other (EN 60601-1-22).

The time for the laser diode is measured by the real-time system. If the maximum time is exceeded, the diode is deactivated and an error is transmitted to the PC system. In addition, the duration of activation of the laser diode is monitored by a suitable hardware circuit. If the diode is activated for an excessive period of time, the laser diode is deactivated by the hardware. The maximum activation duration for each position is 120 seconds.

Pause During Irradiation

The PC system may initiate a pause of the irradiation process. When the real-time system receives a pause command, the irradiation is interrupted and the laser diodes are deactivated. When the therapy is continued, the remaining time is increased by 5 seconds (it should be possible to change the value in the Helbo service menu), and the irradiation of the area continues. (Trigger pause, irradiation is correctly continued).

Safety Functions

A “watchdog” is implemented to prevent program crashes and infinite loops. The program must reset the watchdog at cyclical intervals, otherwise a reset is carried out.

To ensure than an area is not irradiated for too long, the hardware performs automatic shut-off after the laser diode has been activated for longer than 120 seconds.

The laser diode temperature and the diode current are monitored continuously. If a value is outside the valid range, deactivation takes place so as to prevent damage to the laser diodes. When the door contact switch or emergency-off switch is actuated, the laser diodes must be switched off.

If the communication between the PC system and the real-time system is interrupted, the therapy must be aborted.

Cooling

Each laser diode is cooled by a fan. The speed measurement signal for the fan must be monitored. If a fan fails, the treatment is aborted and an error is reported to the PC system.

Operational Readiness

Operational readiness is not reported to the PC system until all laser diodes are at the operating temperature and the fans are operational.

PC System
The PC system is in charge of:
visualizing the camera images
depicting the distance measurement
starting and stopping irradiation
storing the operating times
storing the areas to be irradiated

Screens/Messages/Audio Notices

Screen Displays:

1. welcome screen (optional): off, continue

2. start screen: appears after system start and is used to position the irradiation unit—the live camera image is displayed, overlaid with the distance indicators. Operating elements: back (to the welcome screen), continue (to the therapy screen)

3. therapy screen: the irradiation grid is displayed with the camera image underneath, miniature display of the current distance measurement, progress of irradiation. Operating elements: back (to start screen), scan, start/pause, abort

4. service menu: settings, must not be accessible to the user

An audio pause operating element is to be provided on all screens.

Languages:

German, English—multilingual options should be provided, additional languages should be easy to implement.

Service menu only in English.

Symbols:

All messages should be supported by symbols.

Error messages:

2 error categories for messages:

a) user or therapy error—for error message see respective REQ

b) system or device error, illuminated red error LED, error message: “Device error xxx. Please restart the device. If the error occurs again, please call technical support.”

Audio notices: An audio alarm signal should be emitted for each message.

Distance Measurement—Graphical Representation

The distance measurement data from the real-time system is processed in the PC system, the distance is represented in color and graphically on the display.

The colors of the bars are defined as follows (distances configurable in the service menu):
dark green: distance from ideal distance (100 mm) is ±5 mm (95 mm-105 mm)
light green: distance from ideal distance is ±10 mm (90 mm-110 mm)
bright red: distance from ideal distance is ±20 mm (80 mm-120 mm)
dark red: distance from ideal distance is >20 mm

(The color rendering is to be verified using standard-compliant protective laser goggles!)

The distance indicator is depicted on 2 different input screens:

1. start screen: adjusting the irradiation unit—the live camera image (unprocessed) is shown (carriage with camera and distance sensor at the end position, on which the fixed distance sensor is not mounted), overlaid with the distance indicators. The distance bars are shown in a transparent fashion because only those bars that represent the currently measured distance are shown in solid colors.

2. therapy screen: miniature display of the current distance measurement (fixed sensor and central distance sensor on the carriage) (optional)

Adjusting the irradiation unit: If the irradiation unit is placed over a large wound, the distance at both ends must be in the green range (=distance 9-11 cm, it should be possible to change this distance value in the Helbo service menu). In the case of smaller wounds, it is possible for only one end of the irradiation element to be in the green range. The distance during the adjustment is measured at the central laser diode. During the scan process, the valid ranges are determined (the process can be started when one end of the irradiation element is in the valid range). (comparison of data from the real-time system and the depiction on the display).

Camera Image, Grid, Irradiation Area

The PC system processes the camera image, which is then shown on the display. A grid is placed over the image. The size of a grid field (in the irradiation region) is:

length (in longitudinal device axis): 30 mm (corresponds to 5 irradiation positions @ 6 mm)

width: 45 mm

The grid consists of a total of 5×3 fields, whereby the entire image covers an area of 150 mm×105-135 mm.

The size of a field on the screen is approximately half the original size (15×23 mm W×H)—and in any case, as large as possible.

(Compare depiction on the display to wound; fields can only be selected if the distance is OK).

Scan

When the scan key is actuated, the image information is read and the distance from the wound is measured at each irradiation position. Message: “Scan is complete. Please select the fields to be irradiated.”

The areas which, according to the scan, are not within the valid distance range are automatically highlighted with a red X.

Irradiation areas should only be selected if the distance to the laser diode from the wound is in the valid range (50-120 mm, in each case measured at the center of the irradiation field for each laser diode; it should be possible to change all distance values of this REQ in the Helbo service menu). This also allows smaller wounds to be irradiated if the distance cannot be maintained over the entire length of the device. However, the user may also select the sub-areas (X) for which irradiation is not valid.

Start

The PC system can start the irradiation process only if at least one area is selected and the distance from the wound is in the valid range.

Prior to starting the irradiation process, a message “Therapy starts—wear protective goggles!” appears (with the goggles symbol) and an audio signal is emitted. Irradiation is started after a delay of 3 seconds, and the message disappears automatically after 10 seconds (configurable).

The text of the “Start” button is changed to “Pause”: (Start is only possible if at least one area is selected).

Irradiation

The areas that have been selected are transmitted to the real-time system. Each selected area is irradiated for 40 seconds. It should be possible to change this duration in the Helbo service menu. Changes to the areas are no longer possible once the program has been started.

The ongoing therapy and the irradiated areas are visualized on the display, for example by way of a migrating bar, which represents the laser position. Areas that have already been irradiated should be highlighted in color (different red hue).

The remaining irradiation time should be displayed.

(Control the laser position and compare to a selected area).

Continuous Distance Control

If the real-time system reports that distance of a laser diode (applies to all 3) is too small or too great, a notice is shown on the display and the program is switched to the pause mode. The therapy can only be continued when the distance is in the valid range again (80 mm to 120 mm; it should be possible to change the distance value in the Helbo service menu). The monitoring function for the minimum distance is always activated, while for the maximum distance it is only activated when an area was selected for irradiation.

End of Therapy

The real-time system reports the end of therapy as soon as all areas have been irradiated. The PC system emits an audio message and a visual message. All areas are deselected to prevent renewed irradiation.

Text is required on the display for setting forth the message (translations may be required). If the minimum distance was not adhered to (REQ 305) during irradiation, a notice regarding hygiene measures is to also be shown on the display after acknowledging the message “Therapy has ended,” because the patient may have come in contact with the device. This message is to be acknowledged and appears once more before starting the next treatment (after activation, or activation from stand-by).

Text: Patient may have come in contact with the device—please clean particularly thoroughly!

Time-Exceeding

If the real-time system reports a time-exceeding (maximum activation duration of a laser diode), an error is shown on the display, and the current therapy cannot be continued.

Text is required on the display for setting forth the error message (translations may be required). “Device error xxx. Please restart the device. If the error occurs again, please call technical support.”

Protective laser goggle notice: avoid red symbols and instructional text as much as possible!

Pause During Irradiation

The current therapy can be paused using a key on the touch screen.

The text of the “Pause” button is changed to “Start”:

The progress bar is flashing.

An information message sweeps over the grid: “Please ensure that the irradiated body part is still positioned correctly; if not, abort the therapy.”

The PC system likewise switches to the pause mode if the distance from the wound during irradiation is no longer in the valid range.

When the device is pause mode, there are 2 options for continuing: continue the treatment using the start/pause key (distances must be correct again); abort key: (if distances are no longer correct or for other reasons, for example the patient has moved excessively); (interrupt and continue therapy).

Abort

The current therapy can be aborted using a key on the touch screen. The following scenarios are possible:

1. During irradiation, or

2. In pause mode: query “Therapy is in progress. Are you sure you want to abort?, if aborted: “Therapy has been aborted. Selected area was not fully irradiated!”

3. During display of the therapy screen (same function as “back”)

After the process has been aborted, the device is reset to the original state (start screen). (interrupt and continue therapy).

Operating Times

The following data is stored:
operating time in seconds per laser diode
start of treatment
irradiated fields per treatment
errors
total operating time

The data can be shown on the display in a service menu. The language used for this menu is English.

The service menu is accessed through a special combination on the touch screen.

Safety Functions

Communication with the real-time system is assured by a transmission protocol. If the connection fails or the real-time system no longer responds, an error is shown on the display.

General Requirements

The standards EN 60601-1, EN 60625-1 are to be observed when developing the housing. Moreover, the fitness for use in electrical medical equipment according to EN 60601-6 is to be verified and validated.

The total weight of the irradiation unit should not exceed 13 kg. Lightweight materials (such as aluminum) should therefore be used. Only components that are absolutely essential should thus be installed in the irradiation unit. Because Of the weight, the PC system should be installed in the equipment cart, with the exception of the touch screen. The housing should be easy to clean; recesses and grooves should be avoided.

Housing

The following components must be integrated in the housing of the irradiation unit:
linear drive and guide frame
laser diodes
control devices for the laser diodes
hardware for the real-time system
touch screen, PC
laser operation indicator
stand-by key

The housing must be protected against liquid penetrating from above. Air vents must not be directed at the wound of the patient or at the patient. A cover (transparent polycarbonate (PC) panel, curved (r=240 mm) along the longitudinal device axis, 2 mm thick) is provided on the lower face to prevent damage.

Handles for positioning the irradiation unit are required laterally on the housing. The irradiation unit is mounted to the articulated arm. It is accommodated by way of the VESA 75 mount and is to be designed so that a rotational movement of the irradiation unit is possible.

The size of the housing is defined by the components that are installed and by the sizes of the linear drive and the lateral guide.

The maximum housing leakage current of 0.5 mA as per EN 60601-1 must be adhered to. The materials that are employed should likewise contribute to the EMC shielding of the system. The housing must be protected against penetration by liquid, so as to protect the electronics.

Linear Guide

It must be possible to move the laser diodes over a length of 15 cm. For this purpose, a suitable drive, such as a stepper motor, comprising an incremental encoder for position determination should be used.

The laser diodes must be mounted so that impact is cushioned.

Guidance of the Lateral Diodes

The lateral laser diodes are rigidly connected to the central diode—at a defined distance. They are pivoted inward by an angle of 20°.

1.-10. (canceled)

11. An apparatus for photodynamic therapy or substantially destroying microorganisms at a wound site to which a photosensitizer has been applied, comprising an irradiation unit comprising at least one light for irradiating the wound site, a camera for recording images of the wound site, a display providing a grid on which the images of the wound site are displayed so that the wound site is divided on the display by the grid into a plurality of wound areas, the display also providing visual indicia for differentiating grid areas corresponding to wound areas where irradiation has been completed from wound areas where irradiation has not yet been effected, a drive unit, and a support and guide structure mounting the irradiation unit for moving of the irradiation unit by the drive unit to a plurality of positions so that the light source irradiates the wound site sequentially at each of the grid areas displayed in the display for which the visual indicia indicates that irradiation is to be effected.

12. The apparatus of claim 11, wherein the support and guide structure comprises a linear guide, the linear guide comprising a guide rail, and a carriage supported by the guide rail for linear movement thereon, the irradiation unit being mounted to the carriage.

13. The apparatus of claim 11, wherein the light source comprises a laser.

14. The apparatus of claim 13, wherein the laser is a laser diode.

15. The apparatus of claim 11, comprising at least two of the light sources, the light sources being aligned transversely to a direction of the movement of the irradiation unit.

16. The apparatus of claim 11, comprising at least two of the light sources, the light sources being aligned substantially parallel to a direction of the movement of the irradiation unit.

17. The apparatus of claim 11, further comprising an operating unit, the operating unit comprising a display and an actual or virtual keyboard.

18. The apparatus of claim 11, wherein the camera is mounted on the supported and guide structure for movement with the irradiation unit.

19. The apparatus of claim 12 wherein the camera is mounted to the carriage for movement with the irradiation unit.

20. The apparatus of claim 11, wherein the support and guide structure restricts movement of the irradiation unit to a first direction, parallel to one of two orthogonal axes of the grid, for irradiation of grid areas sequentially arranged parallel to said first direction and the light sources are sequentially arranged along a line extending in a second direction substantially perpendicular to said first direction, for irradiation of grid areas sequentially arranged parallel to said second direction.

21. The apparatus of claim 11, wherein at least one of the light sources is pivotable or is set in a fixed pivoted position so that an axis of irradiation emitted by said at least one light source defines a predetermined angle with respect to an axis of irradiation emitted by at least one other of the light sources.

22. A method for photodynamic therapy or substantially destroying microorganisms at a wound site to which a photosensitizer has been applied by operating the apparatus of claim 11, the method comprising positioning the irradiation unit over the wound site with the light sources directed at the wound site and with an image of the wound site displayed on the display and subdivided by the display grid into wound areas, observing the visual indicia on the display to distinguish grid areas corresponding to wound areas to be irradiated from other grid areas, and moving the light sources to irradiate sequentially a plurality of thereby distinguished wound areas.

23. The method of claim 20, wherein only wound areas which have not been irradiated during current operation of the apparatus are irradiated.
